(19) United States
(12) Patent Application Publication
(10) Pub. No.: US 2023/0173232 A1
(43) Pub. Date: Jun. 8, 2023

(54) A BALLOON CATHETER ASSEMBLY TO PROVIDE BLOOD IN INFLATABLE CONDITION

(71) Applicants: Yash Paul SHARMA, Chandigarh (IN); Anita SHARMA, Chandigarh (IN); Ayush SHARMA, Chandigarh (IN)

(72) Inventors: Yash Paul SHARMA, Chandigarh (IN); Anita SHARMA, Chandigarh (IN); Ayush SHARMA, Chandigarh (IN)

(21) Appl. No.: 17/913,147

(22) PCT Filed: Sep. 1, 2020
(86) PCT No.: PCT/IN2020/050761

§ 371 Date: Sep. 20, 2022

(30) Foreign Application Priority Data
Sep. 6, 2019 (IN) 201911035997

(51) Int. Cl. A61M 25/10

(52) U.S. Cl. CPC A61M 25/10; A61M 2025/1095; A61M 2025/1079; A61M 2025/105; A61M 2025/0024

(57) ABSTRACT

A balloon catheter assembly is disclosed. A balloon catheter assembly comprising a proximal part, a middle part, an inflatable balloon and a long tip located at proximal end. The distal end comprising a port for inflation and middle part is connecting member extended from the proximal part end to distal part end. The middle part comprises of three lumens which is extended up to long tip (1) the inner most lumen permit use of a guidewire, (2) a middle lumen is for inflation and deflation of inflatable balloon through proximal part and (3) an outer lumen is comprises of plurality of holes. The plurality of holes of outer lumen spacedly located before and after of the inflatable balloon. The outer lumen hole of is expandable upon starching outer lumen allow to pass blood continuously.

### FIELD ON INVENTION

The present subject matter of invention is related to a balloon catheter assembly to provide blood flow in inflatable condition.

### BACKGROUND OF INVENTION

Generally, balloon catheter is used to re-open blocked lumen in human vasculature. Balloon catheter are also used to deliver stents to provide scaffolding. During dilatation of human vasculature or implantation of stent, upon patient condition physician need more time in inflatable condition.

Further angioplasty with Drug Eluting Stents (DES) is most common treatment which is inflation for at least 30 second or more based on patient condition. More inflation time leads to longer time blood flow blockage.

Some patients with cardiogenic shock and severe Left Ventricle Systolic Dysfunction, longer inflation of stent, preferably more than 30 second is required.

However, it is longer duration inflation further leads to incidence of hypotension and serious arrhythmia and shorter duration expansion leads to inadequate stent expansion.

In left main coronary artery blockage, non-viability right coronary artery gives only short duration inflation.

Therefore, there is a need in the art to develop a delivery system which can hold balloon for longer time in inflatable position without hindering blood flow.

### BRIEF DESCRIPTION OF FIGURES

The accompanying figures and like reference numerals refer to identical or functionally similar elements throughout the separate views and which together with the description below are incorporated in and form part of the specification, serve to further illustrate various embodiments and to explain various principles and advantages all in accordance with the invention.

FIG. 1 illustrates a balloon catheter.

FIG. 2 illustrates cross section of middle part (lumen)

### DETAILED DESCRIPTION OF THE INVENTION

Before describing in detail embodiments that are in accordance with the invention, it should be observed that the embodiments reside primarily in a balloon catheter. Accordingly, the components have been described to include only those specific details that are pertinent to understanding the embodiments of the invention so as not to obscure the disclosure with details that will be readily apparent to those of ordinary skill in the art having the benefit of the description herein.

In this document, the terms “comprises”, “comprising” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Further, before describing in detail embodiments that are in accordance with the invention, it should be observed that all the scientific and technical terms used herein for describing the invention have same meanings as would be understood by a person skilled in the art.

Generally speaking, pursuant to various embodiments, a balloon catheter assembly used as medical device is disclosed.

Several specific embodiments of the balloon catheter of the present invention will be described hereinafter in greater detail in connection with schematic drawings of the catheter construction. To the extent that the components and features of the catheters in the various embodiments of the present invention correspond, they will retain the same reference numerals from Figure to Figure and will not be separately explained.

Examples of constructions, materials, dimensions and manufacturing processes are provided for selected elements. All other elements employ that which is known to those skilled in the field of the invention. Those skilled in the art will recognize that many of the examples provided have suitable alternatives which may also be utilized.

The catheter system of the present invention may take the form of any balloon catheter used in a variety of medical procedures. For example, the catheter may take the form of a single-operator-exchange (SOE), fixed-wire (FW) or over-the-wire (OTW) type balloon catheter and may be used in coronary, peripheral, cerebral, and other vascular procedures, in addition to urethral and other non-vascular procedures. Other features such as perfusion and drug delivery may also be incorporated into the catheter system. For the purpose of the following discussion, the exemplary embodiments are directed to a catheter system which is particularly suitable for PTCA procedures. However, with simple modifications in construction, the catheter system of the present invention may be used for other medical applications not fully discussed herein.

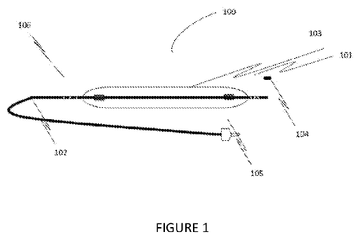

FIG. 1 illustrates balloon catheter assembly (100). An apparatus 100 includes a proximal part (101), a middle part (102), an inflatable balloon (103) and a long tip (103) located at proximal end, inflation port (105) and holes (106).

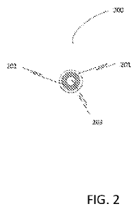

FIG. 2 illustrates cross section of middle part (200), the inner most lumen (203), a middle lumen (202) and an outer lumen (201).

Various embodiment of the invention provides a balloon catheter assembly (100), with a proximal part (101), a middle part (102), an inflatable balloon (103) and a long tip (103) located at proximal end wherein the distal end comprising a port for inflation (105) and the middle part comprises of three lumens (200) extended up to long tip (103), the inner most lumen (203) permit use of a guidewire, a middle lumen (202) is for inflation and deflation of inflatable balloon through proximal part.

An outer lumen (201) is comprises of plurality of holes wherein plurality of holes (106) of outer lumen spacedly located before and after of the inflatable balloon.

Radiopaque markers 107 are provided inside each of the balloons on the catheter shaft 106. These radiopaque markers, which may advantageously be made of metal or other radiopaque material, are preferably longitudinally located on the catheter shaft in the center of each of the balloons for accurate positioning of balloons can be ascertained through fluoroscopy.

In one embodiment, outer lumen hole of is expandable upon starching outer lumen allow to pass blood continuously.

In one another embodiment, outer lumen is elastic polymeric material.

In yet another embodiment, a stent is mounted on expandable member.

In yet another embodiment, the stent is Drug Eluting stent and bare metal stent.

In yet another embodiment stent material is 316 stainless steel, cobalt-chromium alloy and platinum chromium alloy.

In yet another embodiment drug eluting stent is sirolimus eluting stent, anti-proliferative drug eluting stent.

The one or more drugs are selected from a group, including, but not limited to, anti-restenotic agent, an anti-proliferative agent, an anti-inflammatory agent, an antithrombotic agent, and an antioxidant an immunosuppressive agent, a cytostatic agent and a cytotoxic agent. More specifically, the one or more drugs are selected from a group, including, but not limited to, sirolimus, tacrolimus, paclitaxel, beta-estadiol, rapamycin, everolimus, ethylrapamycin, zotarolimus, ABT-578, Biolimus A9 and analogs of rapamycin mitomycin, myomycine, novolimus, permirolast potassium, alpha-interferon, bioactive RGD and salts, esters or analogues thereof.

In another exemplary embodiment, the drug may include, for example, but not limited to, one or more of sirolimus, tacrolimus, paclitaxel, heparin, beta-estadiol, rapamycin, everolimus, ethylrapamycin, zotarolimus, ABT-578, Biolimus A9, docetaxel and mitomycin.

### Example 1

A rapid exchange multiple lumen balloon catheter assembly is inserted in to human body.
A balloon catheter is attached with inflation device at proximal port (105) and filled with saline for normal saline for standard angioplasty.
Followed by balloon is inserted in vascular site and travelled over guide-wire to blocked lumen. A balloon is inflated at nominal pressure using inflation device.
Upon expansion one lumen will provide pressure to inflate balloon and keep in inflated condition. Another port is allowed blood though holes and allow blood to pass through holes to maintain blood flow.
After appropriate inflation balloon assembly deflated from lumen is pulled back.

Those skilled in the art will realize that the above-recognized advantages and other advantages described herein are merely exemplary and are not meant to be a complete rendering of all of the advantages of the various embodiments of the invention.

In the foregoing provisional specification, specific embodiments of the invention have been described. However, one of ordinary skill in the art appreciates that various modifications and changes can be made to the invention without deviating from the scope of the invention. Accordingly, the provisional specification is to be regarded in an illustrative rather than a restrictive sense, and all such modifications are intended to be included within the scope of the invention.

1. A balloon catheter assembly, comprising:
a proximal part, a middle part, an inflatable balloon and a long tip located at proximal end;
wherein the distal end comprising a port for inflation;
wherein the middle part is connecting member extended from the proximal part end to distal part end;
wherein the middle part comprises of three lumens extended up to long tip, the inner most lumen permit use of a guidewire, a middle lumen is for inflation and deflation of inflatable balloon through proximal part and an outer lumen is comprises of plurality of holes;
wherein plurality of holes of outer lumen spacedly located before and after of the inflatable balloon; and
wherein outer lumen hole of is expandable upon starching outer lumen allow to pass blood continuously.

2. The balloon catheter assembly as claimed in claim 1 wherein the outer lumen is elastic tube.

3. The balloon catheter assembly as claimed in claim 2 wherein the plurality of outer lumen holes are located at least 30 mm before and 10 mm after inflatable balloon.

4. The balloon catheter assembly as claimed in claim 1 wherein middle tube is stainless steel shaft.

5. The balloon catheter assembly as claimed in claim 1 wherein the inflatable balloon is at least dilatation balloon catheter and drug coated balloon catheter.

6. The balloon catheter assembly as claimed in claim 1 wherein outer member of middle part and inflatable balloon is made of silicone, latex, polyisoprene rubber, polyethylene, polyolefin copolymer polyethylene terphathalate, nylon and polyurethanes.

7. The balloon catheter assembly as claimed in claim 1 wherein an angioplasty stent is crimped on the inflatable balloon.
